(19) United States
(12) Patent Application Publication
(10) Pub. No.: US 2005/0221486 A1
(43) Pub. Date: Oct. 6, 2005

(54) Cell culture apparatus and methods

(75) Inventors: WengLong Roy Lin, Newton, MA (US); Ravinder Bhatia, Blue Bell, PA (US); Judith Kadarusman, Catonsville, MD (US); John McLaughlin, Doylestown, PA (US)

Correspondence Address: HUMAN GENOME SCIENCES INC;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 11/087,801

(22) Filed: Mar. 24, 2005

Related U.S. Application Data
(60) Provisional application No. 60/608,918, filed on Mar. 25, 2004.

(51) Int. Cl. C12N 5/02

(52) U.S. Cl. 435/404

(57) ABSTRACT

A method for culturing cells in a plastic culture vessel is provided. The method is suitable for use when it is desirable to have a small hydrophobic molecule present in the cell culture media at some point during incubation.

### CROSS REFERENCE TO RELATED APPLICATIONS

This application claims the benefit of U.S. Provisional Application No. 60/608,918, filed Mar. 25, 2004, which is herein incorporated by reference in its entirety.

### FIELD OF THE INVENTION

The present invention relates to a cell culture apparatus and cell culture methods.

### BACKGROUND OF THE INVENTION

In vitro cell culture is important for many commercial and scientific endeavors, for example, for the production of biopharmaceuticals such as proteins and antibodies. Many devices have been developed for culturing cells, which can be divided into three categories: 1) small-scale devices with culture volumes up to 20 L, typically between 1 L to 20 L, although culture volumes of less than 1 L, even in the range of 10 mL or 100 mL are not exceptional; 2) medium-scale devices with culture volumes between 20 and 2,000 L; and 3) large-scale bioreactors with operating volumes from 2,000 up to 20,000 L. In general, small-scale devices are limited to a few liters in volume because they rely on surface oxygen transfer to provide aeration for cells. Examples of small-scale devices include spinner flasks, T-flasks, and roller bottles. Conventional large-scale bioreactors include stirred tank bioreactors (See, Armstrong et al U.S. Pat. No. 4,906,577 and Morrison U.S. Pat. No. 5,002,890).

Recently, cell culture bags have been developed for use in small- and medium-scale cell cultures (See, for example, Matsumiya et al., U.S. Pat. No. 5,225,346). U.S. Pat. No. 6,190,913 (Singh) describes a cell culture apparatus that includes a gas-permeable cell culture bag placed on a rocking mechanism, which induces a wave-like motion to aerate and maintain the cells in suspension. The WAVE BIOREACTOR™ disclosed in U.S. Pat. No. 6,190,913 patent has comparable performance to stirred-tank bioreactors. Cell culture bags have the advantage of being disposable, which reduces preparation and clean up time. Additionally, cell culture bags are pre-sterilizable, inexpensive, easy to use and require minimal space for storage and use.

Although cell culture bags have been used successfully for the cultivation of many cell types, scientists have had difficulty culturing some cell types, for example cholesterol dependent NSO cells, using cell culture bags. Consequently, scientists have turned to cholesterol independent cells, the use of serum supplements, or back to traditional spinner and shake flasks. However, cholesterol independent cells generally have lower productivity than cholesterol dependent cells. Moreover, serum is expensive and undesirable due to variability in quality, regulatory considerations, and difficulty in removal from the final product. These potential issues with use of serum have pushed the industry to utilize protein-free and chemically defined media alternatives.

### SUMMARY

The invention provides a method for culturing cells in a plastic culture vessel wherein it is desirable to have a small hydrophobic molecule present in the cell culture media at some point during cultivation. Specifically, the plastic culture vessel is constructed from a chemically inert material, such as a fluoropolymer. In one embodiment, the small hydrophobic molecule is added to the cell culture media, for example, if the molecule is important for cell function or vitality. The term “cell function” and “cell vitality” are used interchangeably to refer to at least the following non-limiting examples: protein expression, viability, growth, differentiation, and proliferation, etc. In another embodiment, the small hydrophobic molecule can be added to evaluate an effect of the molecule on cell function or vitality. In yet another embodiment, the small hydrophobic molecule is produced by the cells.

Generally the small hydrophobic molecule has a molecular weight of less than 1,000 g/mol, less than about 500 g/mol, or between about 100 g/mol and about 500 g/mol, and is primarily non-polar, but may contain one or more polar constituents (such as a hydroxyl group, —OH). Hydrophobic molecules generally have low surface tension values, generally less than about 30 mN/m, less than about 20 mN/m, between about 10 mN/m and 20 mN/m, or between about 15 mN/m and 20 mN/m. In one embodiment, the small hydrophobic molecule comprises a terpene. In another embodiment, the terpene is cholesterol.

Fluoropolymers that may be used are known. For example, the fluoropolymer can be selected from the group consisting of polychlorotrifluoroethylene (PCTFE), ethylene-tetrafluoroethylene copolymer (ETFE), fluorinated ethylene-propylene copolymer (FEP), Polytetrafluoroethylene (PTFE), polyvinylidene fluoride (PVDF), perfluoroalkyltetrafluoroethylene copolymer (PFA), tetrafluoroethylene and perfluoromethyl vinyl-ether copolymer (MFA), chlorotrifluoroethylene-vinylidene fluoride copolymer (CTFE/VDF), ethylene-chlorotrifluoroethylene copolymer (ECTFE), polyvinyl fluoride (PVF), and tetrafluoroethylene-hexafluoropropylene copolymer (TFE/HFP). In one embodiment, the fluoropolymer comprises fluorinated ethylene-propylene copolymer (FEP).

In specific embodiments, the cell culture vessel is selected from the group consisting of roller bottle, spinner flask, shaker flask, T-flask, cell culture bag, and plates.

The invention can be used to culture cells such as animal (e.g., mammalian cells), insect, microbial (e.g., bacterial), fungal (e.g., mold and yeast), or plant cells. In one embodiment, the cells comprise cholesterol dependent NSO cells. In another embodiment, the cells comprise hybridoma cells.

### BRIEF DESCRIPTION OF THE DRAWINGS

FIG. 1 shows the chemical structure for cholesterol.

FIG. 2 shows one embodiment of a cell culture vessel according to the invention.

FIG. 3 shows a graph of cell density for NSO cells in medium supplemented with chemically defined cholesterol using cell culture bags of different materials.

FIG. 4A shows the cholesterol concentration in solution in cell culture bags of different materials during pretreatment. Concentration was measured as a percentage of a theoretical value.

FIG. 4B shows the cholesterol concentration in solution in cell culture bags of different materials during different stages of culture in the WAVE BIOREACTOR™.

FIG. 5 shows the viable cell density for NSO cells in medium supplemented with different cholesterol sources using a low density polyethylene (LDPE) cell culture bag.

### DETAILED DESCRIPTION

Cell culture bags have the advantage of being inexpensive and disposable and require minimal space for storage and use. Additionally, cell culture bags are easy to use and can be pre-sterilized, thereby reducing the potential for contamination. Although cell culture bags have been used successfully for the cultivation of many cell types, scientists have had difficulty culturing some cell types, for example cholesterol dependent NSO cells, using cell culture bags.

While not intending to be limited by theory, the difficulty in culturing some cell types in cell culture bags is believed to be the result of interactions of small hydrophobic components present in the culture media with the surface of the cell culture bag. The interactions between the small hydrophobic components and the bag surface are affected by many factors, including but not limited to, side chain hydrophobicity, degree of side chain branching, pore size distribution, and pore shape. In particular, in cell culture bags made from porous material, for example, low-density polyethylene (LDPE), small hydrophobic components can become trapped within the pores. It was discovered that cell culture bags made from chemically inert material, including bags having a chemically inert lining or coating, and laminates, are particularly advantageous for cell cultures that include one or more small hydrophobic components in the cell culture media (for example, cholesterol dependent NS0 cells) or for cells that produce a hydrophobic cell product (for example, TAXOL® producing cells). Although hydrophobic side chains may be present on the chemically inert material, the hydrophobic components do not become trapped within pores of the inert material.

### 1. Hydrophobic Components

Small hydrophobic components include, but are not limited to, nutrients, metabolic products generated by the cell culture, and other additives present in the culture media. As used herein, the term “small hydrophobic components” refers to compounds having a molecular weight of less than about 1000 g/mol, less than about 500 g/mol, or between about 100 g/mol and about 500 g/mol, which are primarily non-polar, but may contain one or more polar constituents (such as a hydroxyl group, —OH). Hydrophobic molecules generally have low surface tension values, generally less than about 30 mN/m, less than about 20 mN/m, between about 10 mN/m and 20 mN/m, or between about 15 mN/m and 20 mN/m.

In some instances it may be desirable to introduce a small hydrophobic component in cell culture media and/or maintain a desired amount of small hydrophobic component in the cell culture media. As used herein, the term “introduce” can refer to both the exogenous addition of the small hydrophobic component, for example by personnel (either manually or in an automated process, for example, using appropriate machinery) and the endogenous addition of the small hydrophobic component due to cellular activity. The introduction of the small hydrophobic molecule to the culture media can occur before or after inoculation of the media with cells.

For example, some cell lines require the presence of a hydrophobic component in the cell media for survival, such as cholesterol-dependent NSO cells. In another instance, it may be desirable to include a hydrophobic component in the culture media to evaluate the effect of the component on cell function or to induce protein expression. In other instances, it may be desirable to recover or measure the amount of hydrophobic metabolic products produced by a cell culture.

The concentration of small hydrophobic molecule in the cell culture media can vary. However, the concentration is typically at least about 0.2 mg/L, between about 0.2mg/L and about 100 mg/L, between about 5 mg/L and about 50 mg/L. Concentration can be determined using known methods, including Gas Chromatography (GC), Reverse Phase-High Performance Liquid Chromatography (RP-HPLC) with Ultraviolet light (UV) and Mass Spectrometry (MS).

One class of small hydrophobic compounds are terpenes (also referred to as polyisoprenoid compounds). Terpenes are generally small (typically having a molecular weight of less than 500 g/mol) hydrophobic compounds made up of unsaturated 5-carbon isoprene units. Isoprenoids are compounds that are formed by polymerization of multiple units of isoprene. Monoterpenoids, such as limonene, are made from two isoprene units, and sesquiterpenoids from three units. These compounds are relatively volatile plant products that are important flavor and aroma components of food. Cyclic diterpenoids and triterpenoids are also widely distributed. Triterpenoids are the basis for compounds such as cholesterol or diosgenin. While higher plants have the greatest variety of isoprenoids, cholesterol and related sterols are important components of biological membranes.

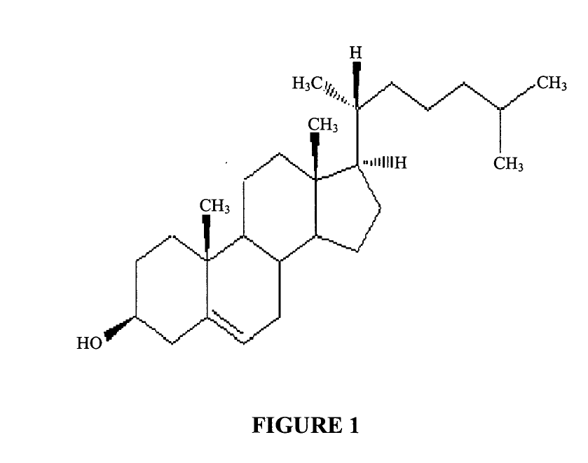

Cholesterol (cholest-5-en-3β-ol), the most abundant member of a family of polycyclic isoprenoids known as sterols, is a terpene (See FIG. 1) that has a molecular weight of about 387 g/mol. Squalene, an intermediate in the synthesis of cholesterol is also a terpene. Steroid hormones, such as cortisol, estradiol, progesterone and testosterone are derived biosynthetically from cholesterol and are modified triterpenes.

Many terpenes have medicinal properties or biological activity. For example, menthol, a monoterpene isolated from various mints, is used as a topical pain reliever and antipuretic (relieves itching). Thujone, another monoterpene, is the toxic agent found in Artemisia absinthium (wormwood) from which the liqueur, absinthe, is made. Borneol is a monoterpene derived from pine oil, and is used as a disinfectant and deodorant. Camphor is a monoterpene that is used as a counterirritant, anesthetic, expectorant, and antipruritic, among many other uses. Gossypol is a dimeric sesquiterpene isolated from the seeds of cotton plants that has been used clinically in China as a male contraceptive. TAXOL® (Paclitaxel), an anti-cancer drug, is a diterpene that was first isolated from the bark of the Pacific yew, Taxus brevifolia. TAXOTERE® (docetaxel) is a TAXOL® analog that has similar activity to TAXOL® (paclitaxel).

Additionally, many anaesthetics are small hydrophobic molecules. Generally, the potency of an anaesthetic depends on its partition coefficient between oil and water. Generally, molecules having a higher solubility in oil have a higher potency. Halothane (2-Bromo-2-chloro-1,1,1-trifluoroethane, C₂HBrClF₃) is a widely used and potent anaesthetic that has a molecular weight of 194.38 g/mol. Other anaesthetics include isoflurane (MW 184 g/mol), enflurane (MW 184 g/mol), desflurane (MW 168 g/mol) and sevoflurane (MW 200 g/mol).

### 2. Chemically Inert Materials

It has been discovered herein that cell culture devices made from chemically inert material, including cell culture devices having a chemically inert lining or coating, or laminates, are particularly useful for cell cultures that include small hydrophobic components in the cell media. As used herein, the term “chemically inert” refers to a material that does not result in a “substantial” or “statistically significant” reduction in the amount of small hydrophobic components in the cell media over time (i.e., between about 1 to about 24 hours, between about 1 to about 10 hours, between about 1 to about 5 hours) in the absence of cellular activity. The term “substantial” reduction in the amount of small hydrophobic component refers to less than about 25%, 15%, 10%, 5% or 1% reduction in the amount of small hydrophobic component over time. The term “statistically significant” refers to the confidence level in the data. As used herein, the term “statistically significant” is defined as having 95% confidence level. “In the absence of cellular activity” can mean, for example, when the media is incubated in the culture vessel without being inoculated. Reduction of the amount of hydrophobic component in the media can easily be determined using known methods, for example by determining the concentration of the small hydrophobic component in the media over time, for example, using gas chromatography (GC) and comparing the change in concentration over time to a control media, for example, media in a glass culture vessel. The term “amount” as used herein can refer to the concentration of the small hydrophobic component as determined by Gas Chromatography (GC) or Reverse Phase-High Performance Liquid Chromatography (RP-HPLC) with Ultraviolet light (UV) and Mass Spectrometry (MS) detection.

While not intending to be limited by theory, it is believed that the amount of small hydrophobic components present in a cell culture media incubated in a cell culture bag may be reduced over time due to entrapment of the hydrophobic component within pores present in the polymer material. The mechanism for depletion of small hydrophobic molecules is thus proposed to be a series of physical interactions, rather than a chemical degradation. The physical interactions are believed to include: (1) attraction of small hydrophobic molecules to the polymeric surface through hydrophobic interactions; (2) diffusion of the small hydrophobic components at or near the bag surface into pore structures on the polymer surface; and (3) entrapment of the small hydrophobic components within the pore structure. Two main types of pores are generally present in polymer material: “ink-bottle” and V-shaped pores (Shaw et al. Introduction to colloid and surface chemistry, 4^{th} Ed. 1991. Butterworth Hinemann). Ink-bottle pores have narrow necks and relatively wide interiors, whereas V-shaped pores have wide necks and relatively narrow interiors. When a small hydrophobic molecule enters an ink-bottle pore, it becomes entrapped and can remain there indefinitely. In contrast, a small hydrophobic molecule, upon entering a V-shaped pore, can enter and exit the V-shaped pore reversibly and thus remains available to the cells in the media. The size and shape of the pores present in a polymer material can vary depending on many factors. For example, the degree of polymer branching (e.g., branching location and frequency) can affect pore shape. The degree of branching can also affect polymer density. A high degree of branching generally results in a lower density, as in the case of ULDPE, whereas a low degree of branching generally results in a higher density, as in the case of LLDPE and LDPE. Generally, polymer materials having a lower density (e.g., less than about 0.912 gram per cubic centimeter (g/cc) in the case of ULDPE) have a larger pore size and are less likely to trap small hydrophobic molecules within their pores when compared to a polymer material containing the same polymer at a higher density. This could possibly be attributed to the pores in the lower density polymer material having relatively larger necks. Generally, the polymer material has a pore size distribution in the Angstrom (or 10 E-10 m) range, typically with a diameter of less than about 50 Å, more typically less than about 10 Å. The surface tension of the polymer material may also affect the interaction between the small hydrophobic molecule and the polymer material. The surface tension of the polymer material is generally less than about 45 mN/m, less than 40 mN/m.

Chemically inert materials suitable for use in a cell culture vessel include perfluorinated plastics, also called fluoropolymers. The term fluoropolymer refers to a family of polymers that contain fluorine. A fluoropolymer can be a homopolymer or a copolymer. Typically, a fluoropolymer is a fluorocarbon resin, more typically a fluorocarbon analog of ethylene such as polytetrafluoroethylene, polymers of chloro-trifluoroethylene, fluorinated ethylene, etc. It is believed that the presence of large fluorine elements in the fluoropolymers block absorption of the hydrophobic component by the polymeric material due to steric hindrance. Because the large fluorine molecules prevent small hydrophobic components from becoming trapped within pores present on fluoropolymeric material, fluoropolymeric material can be considered “chemically inert.” Fluoropolymeric material with a high degree of branching, a lower density, or V-shaped pores are less likely to trap small hydrophobic molecules within their pores. It is believed that small hydrophobic molecules that bypass the fluorine steric hindrance will be able to diffuse back into the media.

Examples of fluoropolymers that can be used in connection with the invention include, but are not limited to, polychlorotrifluoroethylene (PCTFE), ethylene-tetrafluoroethylene copolymer (ETFE), fluorinated ethylene-propylene copolymer (FEP) (also called tetrafluoroethylene-perfluoropropylene; common tradenames include Daikin NEOFLON®, Dupont TEFLON®, and Hoechst HOSTAFLON®), Polytetrafluoroethylene (PTFE), also available from Dupont under the tradename TEFLON®, polyvinylidene fluoride (PVDF), perfluoroalkyltetrafluoroethylene copolymer also known as perfluoroalkoxy (PFA), tetrafluoroethylene and perfluoromethyl vinyl-ether copolymer (MFA), chlorotrifluoroethylene-vinylidene fluoride copolymer (CTFE/VDF), ethylene-chlorotrifluoroethylene copolymer (ECTFE), polyvinyl fluoride (PVF), tetrafluoroethylene-hexafluoropropylene copolymer (TFE/HFP). Fluoropolymers such as PTFE and FEP are particularly well suited for use in connection with the invention. Because FEP is easier to manipulate during manufacturing than PTFE, FEP may be more preferred. Methods for manufacturing vessels using fluorocarbon materials are known and described, for example, in U.S. Pat. Nos. 4,847,462, 4,945,203 and 5,041,225.

Ethylene vinyl acetate (EVA) is another chemically inert polymeric materials that may be suitable for manufacturing cell culture devices, including devices having a chemically inert lining or coating, or laminates.

### 3. Cell Types

The invention is suitable for use with a variety of cell types, including animal, insect, microbial, fungal and/or plant cells. Examples of animal cells include mammalian cells, for example, human (including 293, W138, PER.C6 and Bowes melanoma cells), mouse (including 3T3, NSO, NS1, Sp2/0), hamster (CHO, BHK), monkey (COS, FRhL, Vero), and hybridoma cell lines. Bacterial cells include E. coli, Streptomyces and Salmonella typhimurium cells. Fungal cells include molds (e.g. Aspergillus spp.) and yeast cells such as Saccharomyces cerevisiae and Pichia pastori. Insect cells include Drosophila S2 and Spodoptera Sf9 and Sf21 cells. The cells can be cultured in suspension or can be anchorage dependent.

Appropriate culture mediums and conditions are known. Examples of suitable culture media include, but are not limited to, CD Hybridoma, Dulbecco's Modified Eagle Medium (DMEM), Iscove's Modified Dulbecco's Media (IMDM), Roswell Park Memorial Institute (RPMI), Ham's F12, etc.

In a specific embodiment, the invention is suitable for culturing cholesterol dependent NSO cells. NSO cells are suspension cells commonly used as host cells for production of recombinant proteins. Although NSO cells need an exogenous supply of cholesterol for cell growth, NSO cells can be adapted to grow in serum-free, protein-free cultures in large scale bioreactors, making them attractive for industrial use.

### 4. Cell Culture Vessel

For ease of discussion, the disclosure has focused on the use of cell culture bags. However, the invention is not limited to use with cell culture bags. It can be applied to any plastic cell culture vessel, including but not limited to, cell culture bags; roller bottles; shaker flasks; T-flasks; and culture dishes, including multi-well plates. As used herein, the term “plastic cell culture vessel” refers to culture vessels constructed from a plastic material in their entirety, or in part, and/or culture vessels that include a plastic lining or coating, or laminates. As used herein, the term “plastic” refers to a glass of synthetic or semisynthetic materials that can be molded or extruded into objects or films or filaments.

The invention can be used in connection with cell cultures of any volume, including but not limited to, small-scale cell cultures having a volume of less than about 20 L, including cell cultures having a volume of less than about 1 L (e.g., 25 mL to 100 mL), or from about 1 L to about 20 L, between about 1 L to about 10 L, or between about 1 L to about 5 L, medium-scale cell cultures having a volume between about 20 L and about 2,000 L, or between about 20 L and about 500 L, or between about 100 L and about 500 L, or in connection with large-scale bioreactors having a volume up to about 20,000 L, or between about 1,000 L and about 20,000 L, between about 1,000 L and about 10,000 L, or between about 2,000 L and about 10,000 L.

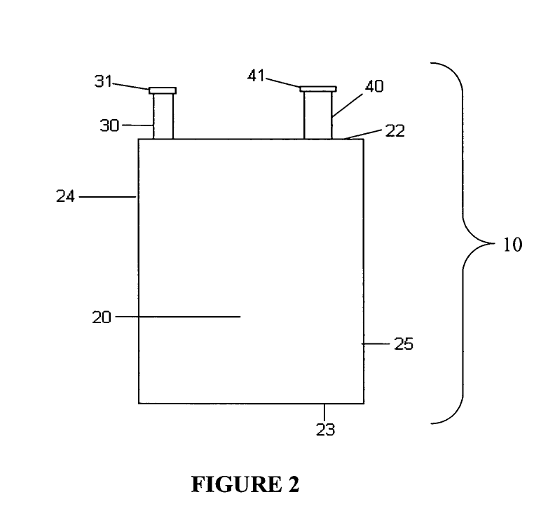

One embodiment of a cell culture vessel according to the invention is shown in FIG. 2. The culture vessel shown in FIG. 2 is a cell culture bag 10. Many configurations for cell culture bags are known and can be routinely used according to the methods of the invention. Generally, the cell culture bag 10 includes a front 20, a back (not shown), a top 22, a bottom 23 and first 24 and second 25 sides. The front 20 and back (not shown) are sealed together by known processes, for example thermal heat sealing. Typically the cell culture bag 10 further includes a sealable inlet port 30 for introducing materials into the culture bag, for example, culture media and cells. The inlet port 30 is constructed such that it can be easily sealed, for example, using a removable sealing mechanism, such as a removable cap 31, or using a permanent sealing mechanism, such as by thermal heat sealing. A variety of sealing mechanisms are known and can be used according to the methods of the invention. The cell culture bag 10 may also include an access port 40 for accessing contents of the bag 10. Typically, the access port 40 is sealed using a removable seal, such as a removable cap 41. A variety of appropriate sealing mechanisms are known and can be used according to the methods of the invention. In one embodiment, the cell culture bag 10 is used in connection with a WAVE BIOREACTOR™ as described in U.S. Pat. Nos. 6,190,913 and 6,544,788.

From the foregoing detailed description, the invention has been described in a preferred embodiment. Modifications and equivalents of the disclosed concepts are intended to be included within the scope of the invention and appended claims. All references cited herein are hereby incorporated by reference in their entirety.

### Working Examples

Cholesterol-dependent NSO cells had low cell growth in low density polyethylene (LDPE) cell culture bags using commercial available media supplemented with chemically defined cholesterol. Various experiments were performed to determine what may have attributed to the lack of growth, including, evaluation of bag material (described in Example 1 —it was determined that some bag materials supported cell growth better than LDPE bags); evaluation of cholesterol source (described in Example 2 —it was determined that only serum supplement supported growth in LDPE bags), cell line specificity (comparing growth between cholesterol-dependent NSO cell line to non-cholesterol dependent NSO cell line—it was determined that the former could not grow in LDPE bags); effects of other components in the media, for example, leachables from the bag material, or suboptimal operating conditions (attempts to support growth by modification of temperature, aeration strategy, rocking rate and speed, did not result in growth in the LDPE bag). The results of these experiments demonstrated that chemically defined cholesterol was depleted from the cell media and thus unavailable for the cells. It is believed that the cause of the cholesterol depletion was not due to chemical degradation, but rather due to cholesterol being removed from the media as a result of the interaction between cholesterol and the bag material.

### Example 1

### Evaluation of Bag Material

Different bag materials were tested and cholesterol depletion from the media in each was quantified. Low-density polyethylene (LDPE) and linear low density polyethylene (LLDPE) bags are commonly used in medical applications for blood collection and handling of biological fluids. Polypropylene (PP) bags have been used for storage. Fluorinated ethylene-propylene copolymer (FEP) bags are used for storage and cell culture. Additionally, an ultra low density polyethylene bag (ULDPE) was also tested. The specifications of bags tested are summarized in Table 1.

Cholesterol-dependent antibody producing, GS-NS0 myeloma cells were cultured in 10 L LDPE bags using the WAVE BIOREACTOR™ system according to manufacturer's instructions (Wave Biotech, New Jersey). Briefly, protein-free, chemically defined CD Hybridoma media (commercially available from Gibco/Invitrogen) was filter-sterilized with 0.22 μm cellulose acetate filter (Coming) and added to the 10 L bags. Prior to inoculation, the media was held in the bag for between 4 to 6 hours (“media hold” time). After the media hold and still prior to inoculation, some of the media was removed for shake flask cultures.

Bag culture: The media remaining in the 10 L bag was seeded with cells to concentrations between 0.3-0.5 E6 cells/mL GS-NS0 cells. The bag was positioned on a 20 L tray holder, and incubated at 37.0° C., 20.0 rpm (increased to 25 rpm Day 3 of culture) and rocking angle 7° until viability decreased to less than 70%. An overlay of air-5% CO₂ was continuously supplied at 0.1 vvm after Day 3. In the first 2 days, the inlet and outlet air ports were clamped and gassed for approximately 20-60 min per day. When the protocol outline above was followed using the LDPE bags, the NS0 cells did not grow. Cell growth was determined by measuring viable cell density (VCD) daily. When VCD did not increase within the first 24-28 hours, it was concluded that the cells did not grow. Shake flask culture: To determine whether the quality of the medium was altered during the media hold; i.e. whether the bag material interacted with the components in the medium, the media removed from the bag was used for shake flask cultures. The shake flasks were inoculated with the same inoculum and cell density as the 10 L bag and maintained in medium +1× cholesterol. Shake flasks with fresh medium containing cholesterol and fresh medium only were run in parallel as positive and negative controls, respectively.

Analytical Assays: Samples were taken daily from the cell culture bags and shake flasks. pH, pCO₂, pO₂ were measured using a blood-gas analyzer (Bayer). Glucose, lactate, glutamate, and glutamine were measured enzymatically (YSI, Yellow Spring). Viable cell concentration and percent viability were determined using the trypan blue exclusion method (Cedex). Antibody product concentration was determined using protein A column on an HPLC system (Waters). All instrumentation was calibrated and operated according to manufacturer's instruction. Cholesterol concentration was measured using solid phase extraction and gas chromatography (GC) (Media Analytical Services, Invitrogen).

Pretreatment: To test whether cholesterol was depleted from the liquid phase, bags were pretreated with excess chemically defined cholesterol (CDC) in 2 L phosphate buffer saline and incubated 5 to 16 hours using the WAVE BIOREACTOR™ system. Cholesterol concentration was reported as percentage of theoretical concentration. Since actual cholesterol concentration was unknown, theoretical concentration was considered as the average measurement of samples containing known multiples of 1× CDC. NS0 cells were then cultured in the LDPE bag (from Wave Biotech) using the same protocol as described above.

Media hold: After pretreatment of bags with excess cholesterol, CD Hybridoma growth media supplemented with cholesterol (1×-5× of amount sufficient for cell growth) was added to the bags and held for 4-6 hours. The amount of cholesterol present in the media was then quantified.

The experiment described above was repeated using linear low-density polyethylene (LLDPE) (Charter Medical), ultra-low-density polyethylene (ULDPE) (Wave Biotech), polypropylene (PP) (Charter Medical) and fluorinated ethylene-propylene copolymer (FEP) (American Fluoroseal Corp.).

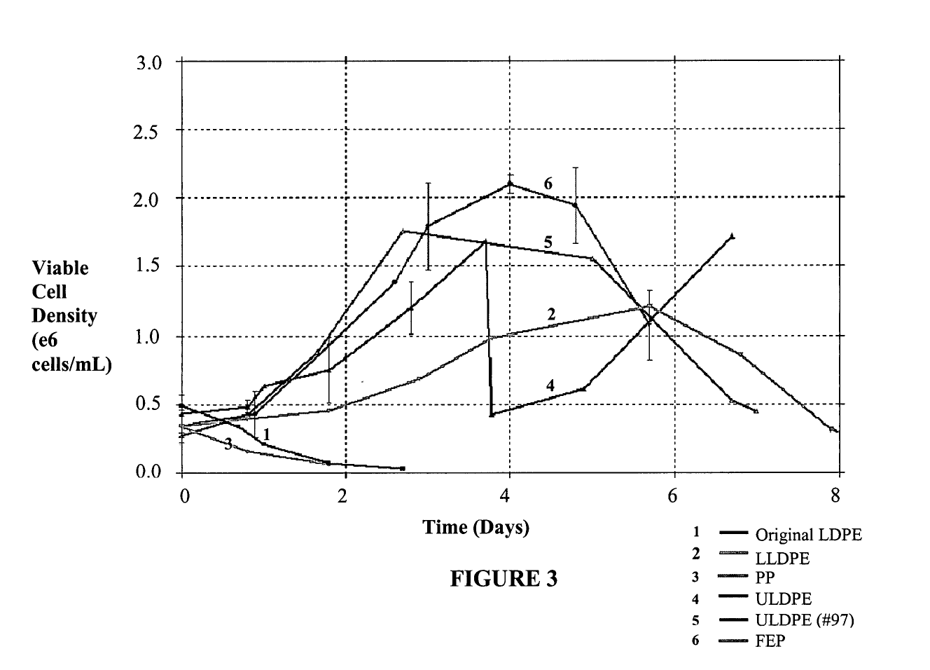

As shown in FIG. 3, cell growth in FEP bag was the highest, followed closely by growth in ULDPE bag. Growth in LLDPE was less than half of the previous two. Cells did not grow at all in the original LDPE and in polypropylene bags (PP). The peak viable cell density and length of cultures is dependent on operating conditions and external feed (when applicable).

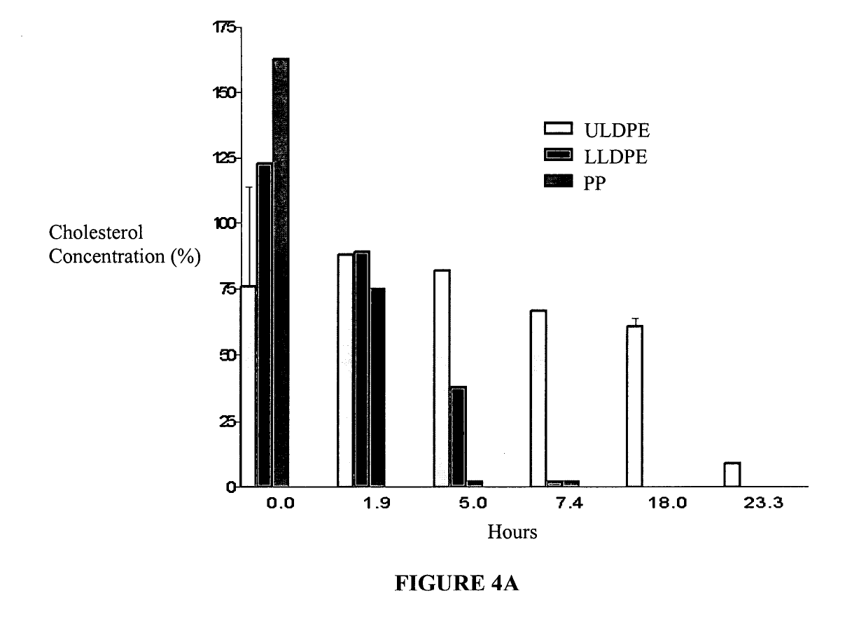

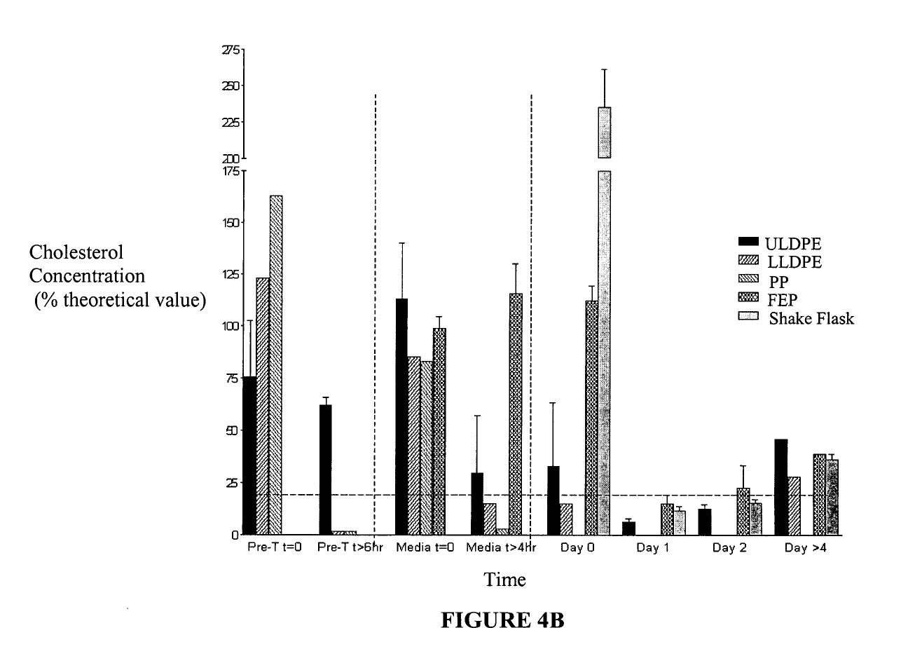

FIG. 4A shows the cholesterol concentration in LLDPE, ULDPE, and PP bags during pretreatment, where only phosphate buffer saline and cholesterol was present in the solution. The cholesterol concentration in the solution decreased for all bags within the first 7 hours, with the lowest decrease in the ultra-low density polyethylene (ULDPE) bag. This result implies that the density of the material (LDPE) might have an effect on the interaction with cholesterol, although this is not a linear relationship (i.e., cholesterol decrease is not proportional to the density of the material). This is consistent with the suggestion that although density may affect pore size distribution, it does not affect pore shape to the same extent. FEP bags were not pretreated due to the assumption that it was not necessary (confirmed in results in FIG. 4B). The same trend was observed for the media hold stage (FIG. 4B). Cholesterol concentration drastically decreased in most bags after only 4 hours of incubation. In ULDPE bags, the concentration decreased, but remained sufficient to support cell growth. In contrast, cholesterol concentration in the media in FEP bags remained constant during media hold, and was more than sufficient to support cell growth. During culture, cholesterol concentration did not remain constant. However, the amount of cholesterol present did support cell growth, as indicated in FIG. 3.

TABLE 1
Comparison of bag polymer material

| | | | Surface tension |
|---|---|---|---|
| | | | γ_{c} at 20° C. |
| | Density (g/cc) | Branching | mN/m |
| LDPE | Low (0.912-0.935) | Yes | 31-33 |
| LLDPE | Linear low (0.935-0.96) | Less than LDPE | ˜33 |
| ULDPE | Ultra low( <0.912) | More than LDPE | ˜31 |
| PP | | Yes | 29 |
| FEP | | Yes | 18-22 |

### Example 2

### Evaluation of Cholesterol Source

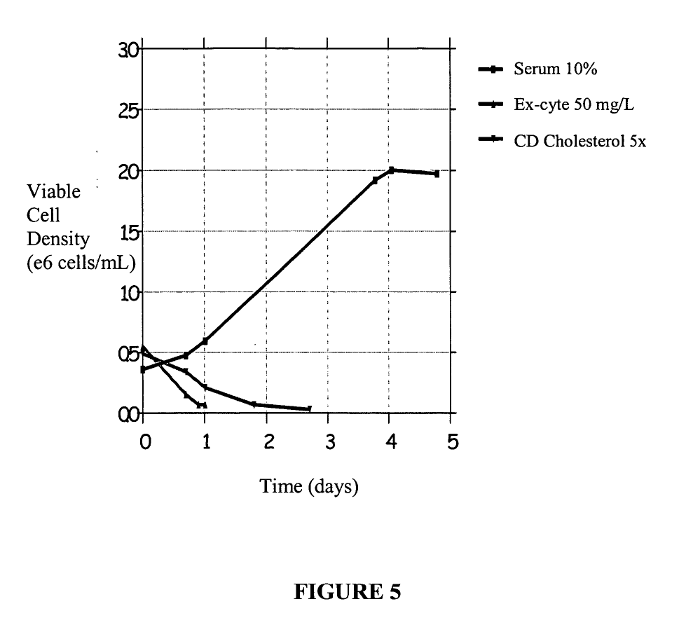

Different cholesterol sources have been shown to effect NS0 cell growth (Gorfien, S., Paul, B., Walowitz, J., Keem, R., Biddle, W. and Jayme, D. Growth of NS0 cells in protein-free, chemically defined medium. Biotechnol Prog 2000, 16(5): 682-7). Therefore, three different cholesterol sources were evaluated to determine their effect on cell growth in LDPE cell culture bags. The experiment was conducted using the protocol described in Example 1, with modifications as indicated below. The results are shown in FIG. 5.

Chemically defined cholesterol (CDC) (commercially available from Gibco/Invitrogen) was diluted to 1×-5× the concentration recommended by the manufacturer and added to the CD Hybridoma medium. The cells did not grow in the LDPE culture bag when supplemented with CDC. However, the same cell line has been known to grow successfully in other vessels, such as glass and stainless steel bioreactors, shake flasks, and T-flasks, when using the same media, same supplements, and same seeding concentration. The results of the experiments demonstrate that the lack of growth in the bag was due to the interaction between the LDPE bag material and CDC.

In another experiment, Ex-cyte™ (Serologicals), a bovine-derived cholesterol lipid supplement, was used in place of cholesterol, at a concentration of 50 μg/mL. The cells did not grow in the LDPE cell culture bag when supplemented with Ex-cyte, even in shake flasks cultures (data not shown). It was suspected that either cholesterol in Ex-cyte was not in a usable form, or that the particular cell line was not compatible with components in Ex-cyte.

In another experiment, Iscove's Modified Dulbecco's Medium (Commercially available from JRH) was used, supplemented with minimal essential amino acids, GS Supplement, and 10% fetal bovine serum. Serum is rich in proteins such as growth hormones, membrane precursor proteins and lipid carrier proteins. Some lipid carriers include low- and high-density lipoproteins (LDL and HDL) and albumin, all of which bind cholesterol. The serum supplemented media supported cell growth in the LDPE cell bag. The presence of serum components such as transport factors (e.g., albumin and transferring, growth factors (e.g., PDGF and EGF) and hormones (e.g., insulin and hydrocortisone), may have aided cell growth in the LDPE cell culture bag.

1. A method for culturing cells in a plastic culture vessel comprising the steps of:
(a) providing a plastic culture vessel constructed from a fluoropolymer;
(b) introducing cell culture media into the cell culturing vessel;
(c) introducing a small hydrophobic molecule into the cell culture media;
(d) inoculating the cell culture media with cells; and
(e) incubating the cell culture under suitable conditions for cell growth.

2. The method of claim 1, wherein the step of introducing a small hydrophobic molecule into the cell culture media comprises exogenous addition of the small hydrophobic molecule to the cell culture media.

3. The method of claim 2, wherein the small hydrophobic molecule is selected from the group consisting of:
(a) a small hydrophobic molecule which is important for cell function; and
(b) a small hydrophobic molecule added to the cell culture media to evaluate an effect of the molecule on cell function.

4. The method of claim 1, wherein the step of introducing the small hydrophobic molecule into the cell culture comprises endogenous production of the small hydrophobic molecule by the cells in the culture.

5. The method of claim 1, wherein the small hydrophobic molecule has a molecular weight of less than about 1000 g/mol.

6. The method of claim 1, wherein the small hydrophobic molecule has a molecular weight of less than about 500 g/mol.

7. The method of claim 1, wherein the small hydrophobic molecule comprises a terpene.

8. The method of claim 1, wherein the small molecule comprises cholesterol.

9. The method of claim 7, wherein the terpene is a steroid hormone.

10. The method of claim 9, wherein the steroid hormone is selected from the group consisting of cholesterol, squalene, cortisol, estradiol, progesterone and testosterone.

11. The method of claim 7, wherein the terpene comprises paclitaxel.

12. The method of claim 1, wherein the fluoropolymer is a homopolymer.

13. The method of claim 1, wherein the fluoropolymer is a copolymer.

14. The method of claim 1, wherein the fluoropolymer is selected from the group consisting of:
polychlorotrifluoroethylene (PCTFE), ethylene-tetrafluoroethylene copolymer (ETFE), fluorinated ethylene-propylene copolymer (FEP), Polytetrafluoroethylene (PTFE), polyvinylidene fluoride (PVDF), perfluoroalkyltetrafluoroethylene copolymer (PFA), tetrafluoroethylene and perfluoromethyl vinyl-ether copolymer (MFA), chlorotrifluoroethylene-vinylidene fluoride copolymer (CTFE/VDF), ethylene-chlorotrifluoroethylene copolymer (ECTFE), polyvinyl fluoride (PVF), and tetrafluoroethylene-hexafluoropropylene copolymer (TFE/HFP).

15. The method of claim 1, wherein the fluoropolymer comprises fluorinated ethylene-propylene copolymer (FEP).

16. The method of claim 1, wherein the cell culture vessel is a cell culture bag.

17. The method of claim 1, wherein the cell culture is selected from the group consisting of:
(a) a small-scale cell culture having a volume of less than about 20 L;
(b) a small-scale cell culture having a volume of less than about 1 L;
(c) a medium-scale cell culture having a volume of less than about 1 L and 500L; and
(d) a large-scale cell culture having a volume up to about 20,000 L.

18. The method of claim 1, wherein the cells are selected from the group consisting of: animal, insect, bacterial, fungal and plant cells.

19. The method of claim 18, wherein the animal cells comprise mammalian cells.

20. The method of claim 19, wherein the mammalian cell is selected from the group consisting of human, mouse, hamster, monkey, and hybridoma cells.

21. The method of claim 20, wherein human cells are selected from the group consisting of 293, WI38, PER.C6 and Bowes melanoma cells.

22. The method of claim 20, wherein mouse cells are selected from the group consisting of 3T3, NS0, NS1, and Sp2/0.

23. The method of claim 20, wherein hamster cells are selected from the group consisting of CHO, and BHK.

24. The method of claim 20, wherein monkey cells are selected from the group consisting of COS, FRhL, and Vero.

25. The method of claim 18, wherein bacterial cells are selected from the group consisting of E. coli, Streptomyces and Salmonella typhimurium cells.

26. The method of claim 18, wherein fungal cells comprise yeast cells selected from the group consisting of Saccharomyces cerevisiae and Pichia pastoris.

27. The method of claim 26, wherein fungal cells comprise Aspergillus spp.

28. The method of claim 18, wherein insect cells are selected from the group consisting of Drosophila S2 and Spodoptera Sf9 and Sf21 cells.

29. The method of claim 1, wherein cells comprise cholesterol dependent NS0 cells.

30. The method of claim 29, wherein the small hydrophobic molecule comprises cholesterol.

31. A method for culturing cholesterol dependent NS0 cells in a plastic culture vessel comprising the steps of:
(a) providing a plastic culture vessel constructed from a fluoropolymer;
(b) introducing cell culture media into the cell culturing vessel;
(c) inoculating the cell culture media with NS0 cells;
(d) introducing cholesterol into the cell culture media; and
(e) incubating the cell culture under suitable conditions for cell growth.

32. The method of claim 31, wherein the fluoropolymer is selected from the group consisting of:
polychlorotrifluoroethylene (PCTFE), ethylene-tetrafluoroethylene copolymer (ETFE), fluorinated ethylene-propylene copolymer (FEP), Polytetrafluoroethylene (PTFE), polyvinylidene fluoride (PVDF), perfluoroalkyltetrafluoroethylene copolymer (PFA), tetrafluoroethylene and perfluoromethyl vinyl-ether copolymer (MFA), chlorotrifluoroethylene-vinylidene fluoride copolymer (CTFE/VDF), ethylene-chlorotrifluoroethylene copolymer (ECTFE), polyvinyl fluoride (PVF), and tetrafluoroethylene-hexafluoropropylene copolymer (TFE/HFP).

33. The method of claim 31, wherein the fluoropolymer comprises fluorinated ethylene-propylene copolymer (FEP).

34. The method of claim 31, wherein the plastic culture vessel comprises a cell culture bag.

35. A method for culturing cells in a plastic culture vessel comprising the steps of:
(a) providing a plastic culture vessel constructed from a vinyl acetate copolymer;
(b) introducing cell culture media into the cell culturing vessel;
(c) introducing a small hydrophobic molecule into the cell culture media;
(d) inoculating the cell culture media with cells; and
(e) incubating the cell culture under suitable conditions for cell growth.

36. The method of claim 35, wherein the vinyl acetate copolymer comprises ethylene vinyl acetate.
